(19) United States
(12) Patent Application Publication
(10) Pub. No.: US 2013/0053729 A1
(43) Pub. Date: Feb. 28, 2013

(54) Urine Diversion Device

(71) Applicants: Darinka Stevic-Wages, South Melbourne (AU); Brendan McKeegan, Jan Juc (AU); Phillip Michael McFarlane, Hampton (AU); Paul Charlwood, Collingwood (AU); Luca Abate, Collingwood (AU)

(72) Inventors: Darinka Stevic-Wages, South Melbourne (AU); Brendan McKeegan, Jan Juc (AU); Phillip Michael McFarlane, Hampton (AU); Paul Charlwood, Collingwood (AU); Luca Abate, Collingwood (AU)

(73) Assignee: FWEE PTY LTD, Hampton (AU)

(21) Appl. No.: 13/697,171

(22) PCT Filed: May 11, 2011
(86) PCT No.: PCT/AU2011/000548

§ 371 Date: Nov. 9, 2012

(30) Foreign Application Priority Data
May 11, 2010 (US) 61333580

(51) Int. Cl. A61B 5/20

(52) U.S. Cl. 600/584; 600/573

(57) ABSTRACT

Embodiments relate generally to urine diversion devices for positioning, in use, at least partly within a toilet bowl. The device in some embodiments comprises a body defining a basin and having a collection side and diverting means to divert urine received in the basin toward the collection side; foldable wings coupled to opposed sides of the body and positionable to overlie at least part of respective opposed sides of a toilet seat; and a cradle to hold a urine receptacle and extending from the collection side of the body, the cradle arranged to support the urine receptacle to receive urine diverted toward the collection side.

### TECHNICAL FIELD

The described embodiments relate generally to methods and devices for urine diversion into a collection device. Some embodiments relate to collection of mid-stream urine while some embodiments do not discriminate between initial stream and mid-stream urine. Some embodiments relate to a method of forming a urine diversion device.

### BACKGROUND

For health or diagnostic purposes, it is common for people to be asked to provide a urine sample. In such circumstances, it is common for a person to be given a small cylindrical cup with a screw-on cap and directed to a nearby toilet. While different people may meet with varying degrees of success in catching urine as it is passed, it is not uncommon for there to be some mess due to imperfect positioning of the cup and/or unpredictable trajectories of the urine.

It may therefore be considered to be desirable in some circumstances to enable collection of urine without the person having to hold the cup. Some devices have been created to sit at least partly inside a toilet bowl so that a person urinating while sitting on the toilet seat can direct urine into a device, which channels the urine into a cup held by the device.

In some situations, a person may be requested to provide a midstream sample of urine. This is because the initial stream of urine flushes the urethra and the midstream urine is thus considered to be a purer sample than urine containing the initial stream.

A person may experience logistical difficulties in obtaining a midstream urine sample in a hygienic manner using a normal urine cup and in-toilet collection devices may not be well suited to enabling collection of the midstream urine without also collecting the initial stream.

Further, in-toilet urine collection devices may be relatively bulky or awkward to use, possibly resulting in frustration or misuse on the part of the person attempting to capture his or her own midstream urine.

The described embodiments address or ameliorate one or more shortcomings or disadvantages associated with previous devices and/or methods for urine collection or at least provide a useful alternative thereto.

### SUMMARY

Some embodiments relate to a urine diversion device for positioning, in use of the device, at least partly within a toilet bowl, the device comprising:
a body defining a basin and having a collection side and diverting means to divert urine received in the body toward the collection side;
foldable wings coupled to opposed sides of the basin and positionable to overlie at least part of respective opposed sides of a toilet seat; and
a cradle to hold a urine receptacle and extending from the collection side of the body, the cradle arranged to support the urine receptacle to receive urine diverted toward the collection side.

The body may define fluid retention means located at the collection side to receive and trap an initial stream of urine directed toward the collection side.

Some embodiments relate to a urine diversion device for positioning, in use of the device, at least partly within a toilet bowl, the device comprising:
a body defining a basin and having a collection side and diverting means to divert urine received in the basin toward the collection side;
support means coupled to the body to support the body in a suspended manner within the toilet bowl; and
a cradle to hold a urine receptacle and extending from the collection side of the body, the cradle arranged to support the urine receptacle to receive urine diverted toward the collection side;
wherein the body defines fluid retention means in the collection side to receive and trap an initial stream of urine directed toward the collection side.

The fluid retention means of the device may comprise a fluid capacity of about 30 ml, for example.

The device may further comprise flow direction means to direct mid-flow urine toward the urine receptacle once the fluid retention means receives a capacity amount of the initial stream. The flow direction means may be movable between a first position, in which urine flowing toward the collection side may be directed into the fluid retention means, and a second position, in which urine flowing toward, the collection side may be directed to bypass the fluid retention means.

The fluid retention means of the device may comprise a fluid reservoir, for example. The flow direction means may comprise a hingedly or pivotally movable flap. The flow direction means may also comprise a float to cause the flow direction means to move to the second position once the capacity amount is received.

The diverting means of the device may comprise ribs formed on the body to channel urine toward a central part of the collection side. In use of the device, the collection side may be disposed at a lower position than an opposed rim of the body. This, together with the diverting means, assists in channelling or focussing the fluid toward the urine receptacle.

The body and the cradle of the device may be integrally formed. The cradle and the collection side may define an overflow opening which may be positioned so that urine overflowing the urine receptacle may pass through the overflow opening into the toilet bowl. In use of the device, the cradle may be arranged to extend laterally from the collection side to receive and support a standard urine collection cup and the cradle may be arranged to support the collection cup at an angle to the vertical. The angle of the collection cup to the vertical may be between about 30° and about 60°, for example.

The device may further comprise a lip formed at an edge of the collection side. The lip may be positioned to act as a ramp to divert urine into an opening of the collection cup when the collection cup is supported by the cradle.

The cradle of the device may be supported in relation to the body by at least one support arm. The at least one support arm may comprise two support arms defining a gap therebetween. When the urine receptacle of the device is supported by the cradle in use of the device, the urine receptacle may be positioned between the support arms. The body and the cradle may be formed of a material suitable to be flushed down the toilet. The material composition may be selected to at least partly disintegrate or break down in water. The material may be coated on an upper, urine-receiving side with a coating that disintegrates or breaks down in water more slowly than the main body material. The main body material may comprise corn starch and the corn starch may be coated with shellac on an upper surface of the device that receives the urine. An underside of the device that does not receive urine may be free of a surface coating that would hinder or substantially slow the material disintegrating or breaking down in water-based fluids.

Some embodiments relate to a urine diversion device to be positioned at least partly in a toilet, the device comprising:
a body defining a basin and having a collection side, the basin shaped to direct urine received in the basin toward the collection side;
support elements on the body to support the body in a suspended manner at least partly in the toilet; and
a cradle defined by the body at the collection side to support a urine receptacle to receive urine directed toward the collection side;
wherein the device is disposable in the toilet.

The device may comprise material that is dissolvable or disintegratable in water and water-based liquids. The body and the cradle may be formed of the material. The material may be a corn-starch-based material, for example.

The device may further comprise means to divert an initial stream of urine from entering the urine receptacle. The means to divert may be arranged to allow urine after the initial stream to enter the urine receptacle. The means to divert may comprise a dissolvable barrier. The dissolvable barrier may be configured to dissolve within about 5 seconds, optionally within about 3 seconds, from when it is contacted by the initial stream of urine.

The dissolvable barrier may have a non-linear shape defining an apex. The apex may be centrally positioned to direct the initial stream towards at least one drainage aperture.

The at least one drainage aperture may comprise two apertures, one aperture positioned toward each lateral side of the body.

The body may define at least one drainage aperture to drain excess urine overflowing from the urine receptacle. The at least one drainage aperture may comprise a central aperture defined by the body to allow drainage of overflow from the urine receptacle.

The dissolvable barrier may be integrally formed with the body. The dissolvable barrier may be formed of a corn starch-based material. The dissolvable barrier may have a thickness between about 0.02 mm and about 0.15 mm. Optionally, the dissolvable barrier may have a thickness between about 0.05 mm and about 0.1 mm.

The support elements may each have an adhesive substance on a face thereof to adhere the respective support element to a rim or seat of the toilet. The support elements may comprise opposed first and second support elements, wherein the second support element is longer than the first support element. At least one of the first and second support elements may comprise an indicator of intended positioning of the device in relation to the toilet.

Some embodiments relate to a urine diversion device to be positioned at least partly in a toilet, the device comprising:
a body defining a basin and having a collection side, the basin shaped to allow urine received in the basin to flow toward the collection side;
support elements on the body to support the body in a suspended manner at least partly in the toilet;
a receptacle support at the collection side to support a urine receptacle to receive urine flowing to the collection side; and
a dissolvable barrier element to act as a barrier to urine flowing toward the collection side.

The dissolvable barrier may be configured to dissolve within about 5 seconds, optionally about 2 to 3 seconds, from when it is contacted by the initial stream of urine.

The dissolvable barrier may have a non-linear shape defining an apex. The apex may be centrally positioned to direct the initial stream towards at least one drainage aperture. The at least one drainage aperture may comprise two apertures, one aperture positioned toward each lateral side of the body.

The body may define at least one drainage aperture to drain excess urine. The at least one drainage aperture may comprise a central aperture defined by the body to allow drainage of overflow from the urine receptacle.

The dissolvable barrier may be integrally formed with the body. The dissolvable barrier is formed of a corn starch-based material, for example. The dissolvable barrier may have a thickness between about 0.02 mm and about 0.15 mm, optionally between about 0.05 mm and about 0.1 mm.

Some embodiments relate to a urine diversion device to be positioned at least partly in a toilet, the device comprising:
a body defining a basin and having a collection side, the basin shaped to allow urine received in the basin to flow toward the collection side;
support elements on the body to support the body in a suspended manner at least partly in the toilet;
a receptacle support at the collection side to support a urine receptacle to receive urine flowing to the collection side; and
wherein at least the body is formed of a molded sheet having a thickness between 0.1 mm and about 1.5 mm.

The molded sheet may have a thickness between about 0.1 mm and about 0.4 mm, optionally between about 0.15 mm and about 0.3 mm. The molded sheet may be at least partly dissolvable in water or water-based fluids. The molded sheet may be a corn-starch-based material.

Some embodiments relate to a package comprising the device described above. The package may be sealed to exclude water and water vapour from the package. The package may be free of the urine receptacle. The package may further comprise pictorial instructions for intended use of the device.

Some embodiments relate to a method of forming a urine diversion device, the method comprising:
molding a body from a moldable material, the body defining:
a basin and having a collection side, the basin shaped to allow urine received in the basin to flow toward the collection side, and
a cradle at the collection side to support a urine receptacle to receive urine flowing to the collection side;
cutting the moldable material to define at least one drainage aperture in the body and to define support elements on the body to support the body in a suspended manner at least partly in a toilet.

The method may further comprise extruding a sheet of the moldable material. The sheet may be extruded to have a thickness of between about 0.1 mm and about 1.5 mm, optionally between about 0.1 mm and about 0.4 mm. In some embodiments, the sheet thickness may be between about 0.15 mm and about 0.3 mm.

The method may further comprise cutting away excess sheet material to define an outside edge of the body. The method may further comprise applying an adhesive substance to at least part of each support element to allow the respective support element to adhere to a seat or rim (or other supporting surface) of a toilet.

The moldable material may be at least partly dissolvable or disintegratable in water or water-based fluids.

At least one of the cutting and molding may comprise providing on the support elements an indication of intended orientation for use. The method may also comprise sealing the body in a package to exclude water and water vapour from the package.

### BRIEF DESCRIPTION OF THE DRAWINGS

Embodiments are described hereinafter in further detail and by way of example, the detailed description of which should be read in conjunction with the accompanying drawings, in which:

FIG. 1 is a perspective view of a urine diversion device according to some embodiments, with flow direction means shown in a first position;

FIG. 2 is a further perspective view of the device of FIG. 1, seen from a different angle and showing the flow direction means in a second position and a collection cup positioned to receive urine;

FIG. 3 is a plan view of the device of FIG. 1, shown without the collection cup and with the flow direction means in the second position;

FIG. 4A is a side cross-sectional view of the device of FIG. 1, taken along line A-A of FIG. 3 and showing the flow direction means in the first position;

FIG. 4B is a side cross-sectional view of the device of FIG. 1, taken along line A-A of FIG. 3 and showing the flow direction means in the second position;

FIG. 5A is a plan view of a urine diversion device according to some embodiments;

FIG. 5B is a side view of the device of FIG. 5A, positioned for use;

FIG. 5C is a bottom perspective view of the urine diversion device of FIG. 5A;

FIG. 5D is a top perspective view of the urine diversion device of FIG. 5A;

FIG. 6 is a top perspective view of a urine diversion device according to further embodiments, shown with a urine receptacle spaced from its intended receiving position in a cradle of the device;

FIG. 7 is a bottom perspective view of the urine diversion device of FIG. 6;

FIG. 8 is a top perspective view of a urine diversion device according to further embodiments, shown in a use position in relation to a seat of a toilet;

FIG. 9A is a plan view of the urine diversion device of FIG. 8, illustrating diversion of an initial urine stream;

FIG. 9B is a plan view of the urine diversion device of FIG. 8; illustrating capture of midstream urine after diversion of the initial stream;

FIG. 10 is a bottom perspective view of the urine diversion device of FIG. 8;

FIG. 11 is a side schematic representation of a package containing a urine diversion device according to described embodiments;

FIG. 12 is a top perspective view of a sheet molded to form a body of a urine diversion device according to described embodiments; and

FIG. 13 is a top perspective view of a urine diversion device body cut from the sheet of FIG. 12.

Like reference indicators as between the drawings are intended to indicate like elements, features or functions. The drawings are not to scale and should be considered to be exemplary, for the purposes of illustrating features and functions of the described embodiments.

### DETAILED DESCRIPTION

The described embodiments relate generally to methods and devices for urine diversion into a collection device. Particular embodiments relate to collection of mid-stream urine, while other embodiments do not discriminate. Still further embodiments relate to methods of manufacture of such devices and packages containing such devices.

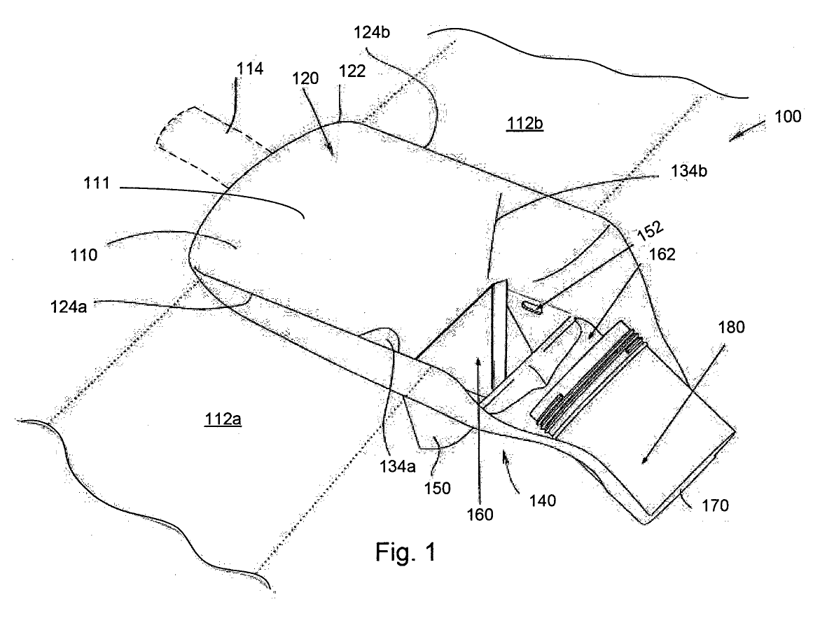

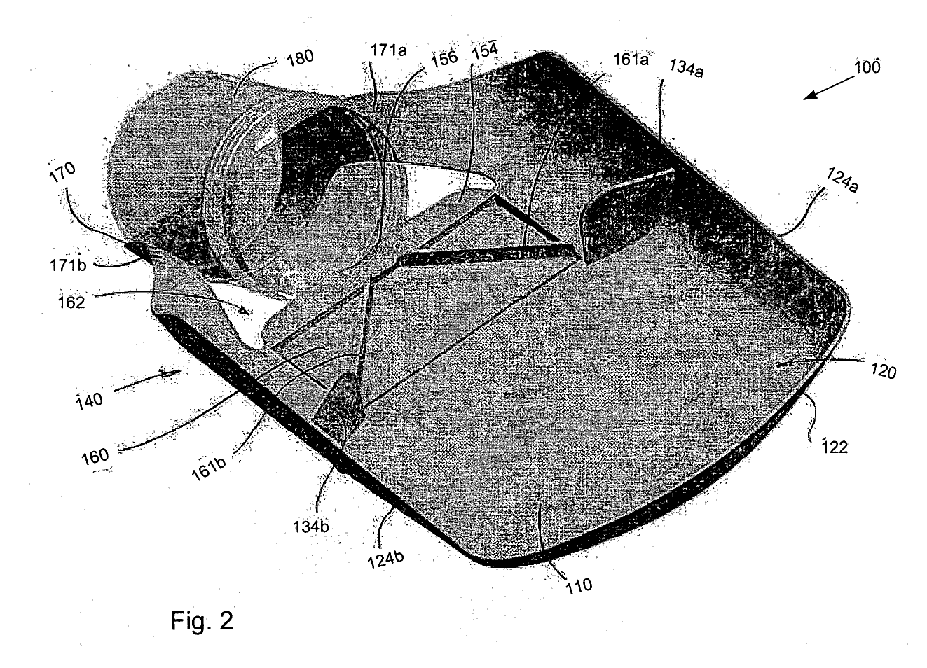

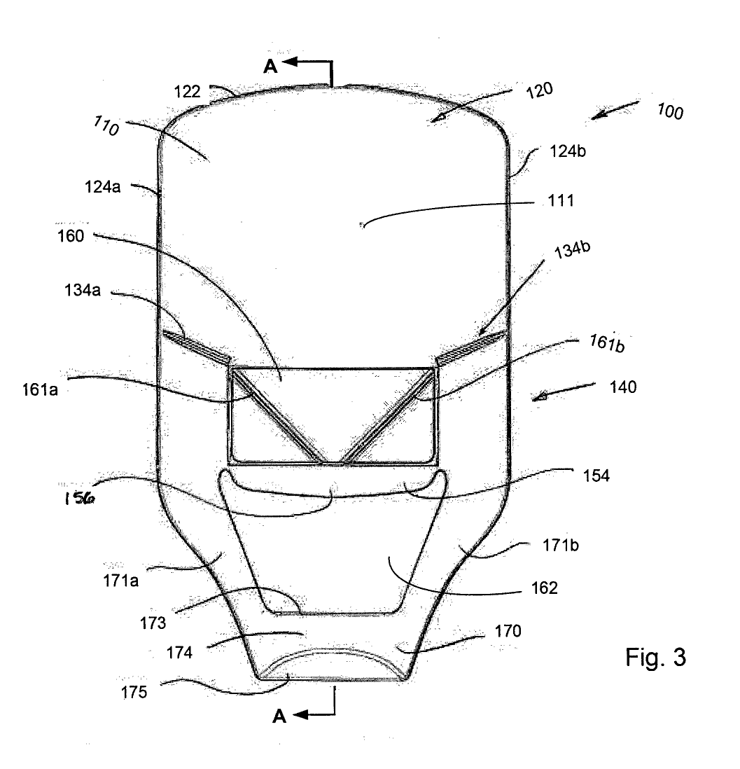

Some embodiments are described by way of example with reference to FIGS. 1, 2, 3, 4A and 4B, all of which show some embodiments of a urine diversion device 100 arranged to divert urine into a standard urine cup 1.80 when the device 100 is positioned to be suspended at least partly within a toilet bowl (not shown) or other form of toilet.

Urine diversion device 100 comprises a body 110 that is shaped to define a basin and has a urine receiving side 120 and an opposed urine collection side 140. Body 110 is generally bowl-shaped on three sides, with upwardly curving side walls extending from a slightly downwardly sloped basin floor 111. The bowl or basin shape of the side walls extends around the urine receiving side 120, but without a corresponding upwardly curved retaining wall at the collection side 140. Instead, collection side 140 is generally arranged to encourage fluid flow from the receiving side 120 into collection cup 180 once an initial fluid volume is received within fluid reservoir 150. A cradle 170 extends from the collection side 140 of body 110 to support collection cup 180 to receive fluid flowing under gravity from collection side 140.

Opposed lateral support wings 112a, 112b may extend from opposed side rims 124a, 124b (or parts adjacent thereto) of body 110. Support wings 112a, 112b are intended to overlie a toilet seat and optionally adhere thereto by an adhesive (not shown) on an underside of the support wings 112a, 112b. Such support wings 112a, 112b may thus provide support to retain body 110 in a suspended position within the toilet bowl while a person is seated on the toilet seat. Support wings 172a, 112b may also provide a hygiene barrier and, in some embodiments, may be sized to cover a substantial area (for example, almost all) of the toilet seat.

In some embodiments, a front support flap 114 may be coupled to body portion 110, with adhesive on a part thereof to secure flap 114 to the toilet seat and assist in positioning and supporting body portion 110 relative to the toilet seat. Flap 114 may be formed centrally along a forward rim 122 of body 110 or to one or both lateral sides along rim 122, for example to account for toilet seats that have a central forward gap that may correspond to a centrally positioned flap 114.

Both support wings 112a, 112b and support flap 114 may be formed of light, flushable materials, such as cellulosic materials, corn starch or other materials that lose structural rigidity or integrity in contact with water-based fluids (i.e. including urine). Light pressure-sensitive adhesive substances that can be easily manually applied and removed may be used to adhere the support wings 112a, 112b and/or support flap 114 to the toilet seat or a part of the outside of the toilet bowl. Support wings 112a, 112b and support flap 114 may be affixed to the body 110 by a suitable adhesive, such as a biodegradable adhesive, for example.

Urine receiving side 120 is generally basin-shaped to receive urine directed toward body 110 when a person is seated on the toilet and device 100 is positioned so that body 110 is generally (but possibly not entirely) positioned within a volume defined by the toilet seat and the toilet bowl. In this position, the cradle 170 will generally be positioned toward a rear part of the toilet (i.e. toward where a cistern is sometimes located) and front rim 122 will generally be positioned toward a front of the toilet (as seen by a person sitting normally on the toilet). In such an arrangement, urine passed from a person sitting on the toilet will normally be received within the basin defined by the outwardly curved walls of urine receiving side 120 and sloped basin floor 111, with those walls tending to direct the urine generally inwardly and (at least in part due to a slope of the basin floor 111 of body 110) toward collection side 140.

Flow direction means, for example in the form of upstanding ribs 134a, 134h, may be at least partly provided on body 110 on the urine receiving side 120 and extending toward urine collection side 140 in order to direct urine inwardly and centrally toward collection side 140.

Receiving side 120, including the side walls, basin floor 110 and ribs 134a, 134b, effectively provides diversion means to divert urine received in the body 110 toward the collection side 140.

Collection side 140 of body 110 comprises fluid reservoir 150 and movable flow direction means, for example in the form of a flap 160. Flap 160 is hingedly movable relative to a lateral line extending across body portion 110 in a transition region from receiving side 120 to collection side 140. Flap 140 is movable between a first (lowered) position, in which it pends downwardly under gravity to be received within-a fluid cavity 153 defined by fluid reservoir 150, and a second (raised) position, in which the flap 160 generally acts as a cover or bridge over the fluid reservoir 150, generally joining (in a fluid flow sense) the urine receiving side 120 with a fluid ramp or lip 154 positioned on collection side 140 to direct fluid into collection cup 180. Flap 160 thus at least partially provides a fluid path from the receiving side 120 through the collection side 140 and into collection cup 180 when flap 160 is in its second (raised) position.

Flap 160 may have a generally flattened upper surface, except for flow direction means, for example in the form of upstanding ribs, walls or guides 161a, 161b, that direct fluid inwardly toward a central part of lip 154 when flap 160 is in its raised position. Flow director ribs 134a, 134b are positioned to provide a roughly continuous inward flow direction in combination with flow guides 161a, 161b when the flap 160 is in its raised position.

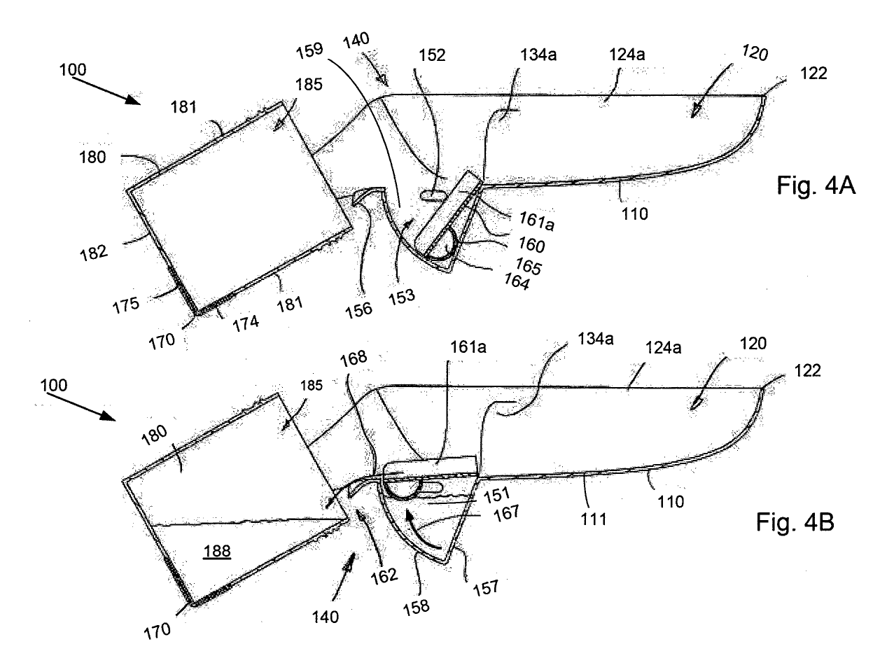

As is visible in FIGS. 4A and 4B, flap 160 has flotation means, for example in the form of a float having an enclosed gaseous volume 164 within a fluid impermeable membrane 165 located on an underside of flap 160. Gaseous volume 164 and membrane 165 are positioned at an opposite end of flap 160 to the end or edge along which flap 160 hinges or pivots, so that, as fluid reservoir 150 fills with fluid, such as urine travelling toward collection side 140 from receiving side 120, the flotation means of flap 160 causes flap 160 to generally rest above the level of the fluid in fluid reservoir 150, thereby raising flap 160 to its second (raised) position as fluid reservoir 150 fills.

Flap 160 is dimensioned to allow a small gap between its non-hinging edge and an adjacent arcuate wall 158 of fluid reservoir 150 so that fluid can pass around the non-hinging edge of flap 160 to be trapped within a containing volume 153 of fluid reservoir 150. This containing volume 153 is defined by the arcuate wall 158 (curved to accommodate the swinging upward motion of flap 160 shown by arrow 167 as it moves towards its raised position) and an opposite second wall 157, as well as opposed side walls 159. Wall 157 may be generally straight or possibly irregularly shaped to accommodate a shape of the flotation means. Wall 157 may be integrally formed with arcuate wall 158 and/or basin floor 111.

Side walls 159 of fluid reservoir 150 each define an overflow aperture 152 to allow overflow of fluid from fluid reservoir 150, so that flap 160 is not raised too high by the level of fluid retained in fluid reservoir 150. Additionally, body 110 may define a stop mechanism (not shown), for example adjacent an upper rim of fluid reservoir 150, to prevent flap 160 from pivoting too far upwardly and thus possibly hindering or misdirecting the fluid flow.

Once flap 160 moves to its raised position, fluid received in receiving end 120 and flowing toward collection side 140 will be directed to cross the flap 160 and run over ramp 154 into collection cup 180 in the manner indicated by arrow 168. Ramp 154 comprises a central spout portion 156 formed to encourage fluid to pour centrally across ramp 154 and through an opening 185 defined by an upper rim of collection cup 180. Fluid reservoir 150 thus captures the initial urine stream, with the midstream urine being directed into collection cup 180. To this end, fluid reservoir 150 is sized to hold a fluid volume 151 of about 30 millilitres before overflowing through overflow apertures 152.

Collection cup 180 may be generally cylindrically formed, with a cylindrical side wall 181 and a base 182 to define an enclosed volume when a cap (not shown) is screwed on over opening 185. Cradle 170 is supported by support arms 171a, 171b extending rearwardly and somewhat downwardly from the collection side 140 and is shaped to receive and support part of the curved side wall 181 and base 182 of collection cup 180. Cradle 170 comprises a curved section 174 that is curved to nestingly accommodate the cylindrical side wall 181 of collection cup 180 and a flat section 175 that is shaped and positioned to support the base 182. Cradle 170 is shaped and positioned to receive and support collection cup 180 generally under a centre of gravity of collection cup 180 while collection cup 180 is empty and as it fills with fluid.

An overflow aperture 162 is defined by cradle 170, support arms 171a, 171b and lip 154 so that, once collection cup 180 fills to the point where urine 188 would overflow the cup's upper rim, that urine can flow over the rim and into the toilet bowl through aperture 162. Cradle 170 is arranged to support collection cup 180 at an angle to the vertical, for example in the order of 70 to 20 degrees from the vertical and optionally between about 3.0 degrees and about 60 degrees to the vertical.

Once the person has finished passing urine, the collection cup 180 can simply be lifted from cradle 170 and the cap screwed on for provision to a health care professional, for example.

Once urine has been passed and the collection cup 180 has been removed from device 100, the support wings 112a, 112b (and support flap 114, if appropriate) can be lifted up from the toilet seat and the entire device 100 conveniently disposed of.

In some embodiments, device 100 may be formed of suitable light materials, including paper and plastics materials, that can be readily hygienically disposed of in normal disposal methods.

In some embodiments, body 110 (including integrally formed receiving side 120, collection side 140 and cradle 170) may be formed predominantly of a material that dissolves, disintegrates or otherwise breaks down in water or water-based fluids, such as may be commonly present in a toilet bowl. Such body materials may include, for example, moulded corn starch-based plastic materials, such as Plantic HP1, made by Plantic Technologies of Victoria, Australia. In such embodiments, an upper surface of body 110 that may come into contact with the urine as it is passed may be coated with a coating that more slowly disintegrates or breaks down than the main body material. Such a coating may include a shellac coating, for example. Body 110 may be uncoated on its underside, for example, where it would not normally come into contact with urine or other water based fluids so that, when device 100 is dropped into a liquid volume at a bottom of the toilet bowl, the material of device 100 begins to break down, so that it can be flushed through the toilet and be treated along with other sewerage.

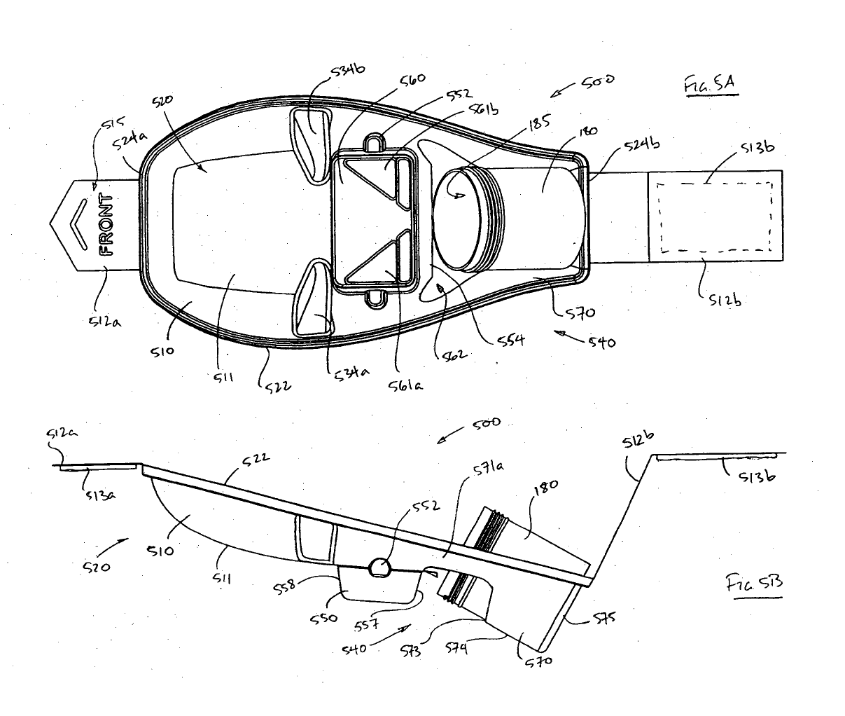

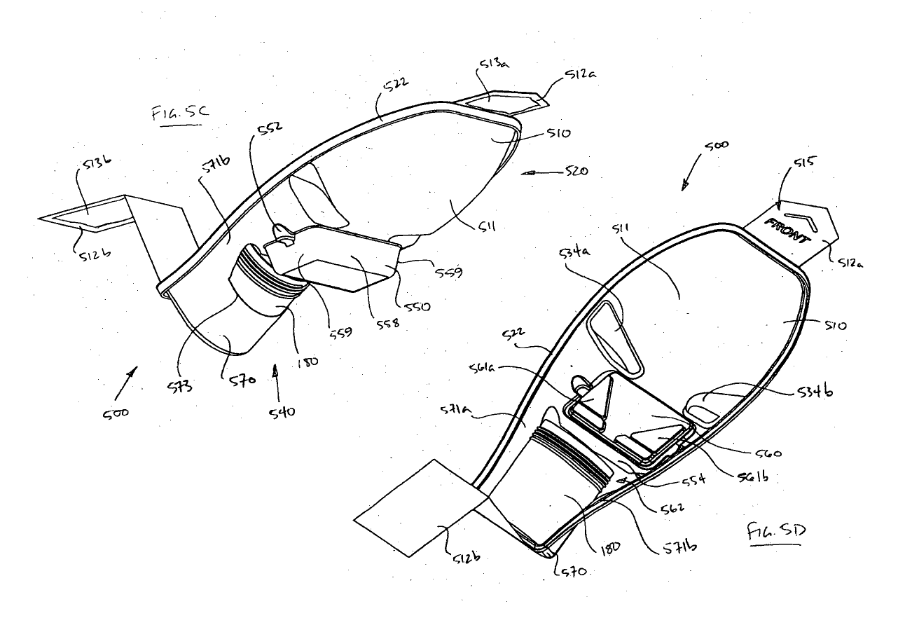

Some embodiments are described by way of example with reference to FIGS. 5A, 5B, 5C and 5D, which show some embodiments of a urine diversion device 500 arranged to divert urine into a standard urine cup 180 when the device 500 is positioned to be suspended at least partly within a toilet bowl (not shown) or other form of toilet. Device 500 is similar to device 100, except that it has a modified shape, different position of support wings and a modified reservoir/flap arrangement.

Urine diversion device 500 comprises a body 510 that is shaped to define a basin and has a urine receiving side 520 and an opposed urine collection side 540. Body 510 is generally bowl-shaped on three sides, with upwardly curving side walls extending from a slightly downwardly sloped basin floor 511. The bowl or basin shape of the side walls extends around the urine receiving side 520, but without a corresponding upwardly curved retaining wall at the collection side 540. Instead, collection side 540 is generally arranged to allow, encourage or direct fluid flow from the receiving side 520 into collection cup 180 once an initial fluid volume is received within fluid reservoir 550. A cradle 570 extends from the collection side 540 of body 510 to support collection cup 180 to receive fluid flowing under gravity from collection side 540.

Opposed front and back support wings 512a, 512b may extend from opposed front and back rims 524a, 524b (or parts adjacent thereto) of body 510. Support wings 512a, 512b are intended to overlie a toilet seat and optionally adhere thereto by an adhesive 513a, 513b on an underside of the support wings 512a, 512b. Such support wings 512a, 512b may thus provide support to retain body 510 in a suspended position within the toilet bowl while a person is seated on the toilet seat.

In some embodiments an additional support flap may be coupled to body portion 510, with adhesive on a part thereof to secure the flap to the toilet seat and assist in positioning and supporting body portion 510 relative to the toilet seat. The additional flap may be formed along a rim 522 of body 510 to one or both lateral sides of a longitudinal centre line of body 510 along rim 522, for example to account for toilet seats that have a central forward gap that may correspond to a centrally positioned flap or support wing 512a. The additional flap may thus be used in tandem with support wing 512a to support body 510 at the front of the toilet.

Both support wings 512a, 512b (and any additional support flap) may be formed of light, flushable materials, such as cellulosic materials, corn starch or other materials that lose structural rigidity or integrity in contact with water-based fluids (i.e. including urine). Light pressure-sensitive adhesive substances that can be easily manually applied and removed may be used to adhere the support wings 512a, 512b and/or a support flap to the toilet seat or a part of the outside of the toilet bowl. Support wings 512a, 512b (and any additional support flap) may be integrally formed with, or otherwise coupled to, the body 510 by a suitable adhesive, such as a biodegradable adhesive, for example.

Urine receiving side 520 is generally basin-shaped to receive urine directed toward body 510 when a person is seated on the toilet and device 500 is positioned so that body 510 is generally (but possibly not entirely) positioned within a volume defined by the toilet seat and the toilet bowl. In this position, the cradle 570 will generally be positioned toward a rear part of the toilet (i.e. toward where a cistern is sometimes located) and a front portion of rim 522 will generally be positioned toward a front of the toilet (as seen by a person sitting normally on the toilet). In such an arrangement, urine passed from a person sitting on the toilet will normally be received within the basin defined by the outwardly curved walls of urine receiving side 520 and sloped basin floor 511, with those walls tending to direct the urine generally inwardly and (at least in part due to a slope of the basin floor 511 of body 510) toward collection side 540.

Flow direction means, for example in the form of upstanding ribs 534a, 534b, may be at least partly provided on body 510 on the urine receiving side 520 and extending toward urine collection side 540 in order to direct urine inwardly and centrally toward collection side 540.

Receiving side 520, including the side walls, basin floor 510 and ribs 534a, 534b, effectively provides diversion means to divert urine received in the body 510 toward the collection side 540.

Collection side 540 of body 510 comprises fluid reservoir 550 and movable flow direction means, for example in the form of a flap 560. Flap 560 is hingedly movable relative to a lateral line extending across body portion 510 in a transition region from receiving side 520 to collection side 540. Flap 540 is movable between a first (lowered) position, in which it pends downwardly under gravity to be received within a fluid cavity 553 defined by fluid reservoir 550, and a second (raised) position, in which the flap 560 generally acts as a cover or bridge over the fluid reservoir 550, generally joining (in a fluid flow sense) the urine receiving side 520 with a fluid ramp or lip 554 positioned on collection side 540 to direct fluid into collection cup 580. Flap 560 thus at least partially provides a fluid path from the receiving side 520 through the collection side 540 and into collection cup 580 when flap 560 is in its second (raised) position.

Flap 560 may have a generally flattened upper surface, except for flow direction means, for example in the form of molded upstanding ribs, walls or guides 561a, 561b, that direct fluid inwardly toward a central part of lip 554 when flap 560 is in its raised position. Flow director ribs 534a, 534b are positioned to provide a roughly continuous and/or additive inward flow direction in combination with flow guides 561a, 561b when the flap 560 is in its raised position.

Flap 560 has flotation means, for example in the form of the material of the float having a density less than urine and tending to float on top of any urine in reservoir 550. Alternatively, a float may be provided, as shown and described in relation to device 100. As fluid reservoir 550 fills with fluid, such as urine travelling toward collection side 540 from receiving side 520, the flotation means of flap 560 causes flap 560 to generally rest above the level of the fluid in fluid reservoir 550, thereby raising flap 560 to its second (raised) position as fluid reservoir 550 fills.

Flap 560 is dimensioned to allow a small gap between its non-hinging edge and an adjacent wall 558 of fluid reservoir 550 so that fluid can pass around the non-hinging edge of flap 560 to be trapped within a containing volume of fluid reservoir 550. This containing volume is defined by a side wall 558 and an opposite second wall 557, as well as a bottom and further opposed side walls 559. Wall 557 may be generally straight or possibly irregularly shaped. Wall 557 may be integrally formed with wall 558 and/or basin floor 511.

Side walls 559 of fluid reservoir 550 (optionally in combination with adjacent upwardly extending side walls of body 510) each define an overflow aperture 552 to allow overflow of fluid from fluid reservoir 550, so that flap 560 is not raised too high by the level of fluid retained in fluid reservoir 550. Additionally, body 510 may define a stop mechanism (not shown), for example positioned adjacent an upper rim of fluid reservoir 550, to prevent flap 560 from pivoting too far upwardly and thus possibly hindering or misdirecting the fluid flow.

Once flap 560 moves to its raised position, fluid received in receiving end 520 and flowing toward collection side 540 will be directed to cross the flap 560 and run over ramp 554 into collection cup 180 in the same manner as indicated by arrow 168 in FIG. 4B. Ramp 554 comprises a central spout portion 556 formed to encourage fluid to pour centrally across ramp 554 and through an opening 185 defined by an upper rim of collection cup 180. Fluid reservoir 550 thus captures the initial urine stream, with the midstream urine being directed into collection cup 180. To this end, fluid reservoir 550 may be sized to hold a fluid volume of about 30 millilitres, for example, before overflowing through overflow apertures 552.

Cradle 570 is supported by support arms 571a, 571b extending rearwardly and somewhat downwardly from the collection side 540 and is shaped to receive and support part of the curved side wall 181 and base 182 of collection cup 180. Cradle 570 comprises a curved section 574 that is curved to nestingly accommodate the cylindrical side wall 181 of collection cup 180 and a flat section 575 that is shaped and positioned to support the base 182. Cradle 570 is shaped and positioned to receive and support collection cup 180 generally under a centre of gravity of collection cup 180 while collection cup 180 is empty and as it fills with fluid.

An overflow aperture 562 is defined by cradle 570, support arms 571a, 571b and lip 554 so that, once collection cup 180 fills to the point where urine 188 would overflow the cup's upper rim, that urine can flow over the rim and into the toilet bowl through aperture 562. In some embodiments, support arms 571a, 571b may be thickened relative to other parts of body 570 for added support for larger collection cups 180. Cradle 570 is arranged to support collection cup 180 at an angle to the vertical, for example in the order of 70 to 20 degrees from the vertical and optionally between about 30 degrees and about 60 degrees to the vertical.

Once the person has finished passing urine, the collection cup 180 can simply be lifted from cradle 570 and the cap screwed on for provision to a health care professional, for example.

Once urine has been passed and the collection cup 180 has been removed from device 500, the front and back support wings 512a, 512b (and any other support flap, if appropriate) can be lifted up from the toilet seat and the entire device 500 conveniently disposed of.

In some embodiments, device 500 may be formed of suitable light materials, including paper and plastics materials, that can be readily hygienically disposed of in normal disposal methods.

In some embodiments, body 510 (including integrally formed receiving side 520, collection side 540 and cradle 570) may be formed predominantly of a material that dissolves, disintegrates or breaks down in water or water-based fluids, such as may be commonly present in a toilet bowl. Such body materials may include, for example, moulded corn starch-based plastic materials, such as Plantic HP1, made by Plantic Technologies of Victoria, Australia.

In such embodiments, an upper surface of body 510, that may come into contact with the urine as it is passed may be coated with a coating that more slowly disintegrates or breaks down than the main body material. Such a coating may include a shellac coating, for example. Body 510 may be uncoated on its underside, for example, where it would not normally come into contact with urine or other water based fluids so that, when device 500 is dropped into a liquid volume at a bottom of the toilet bowl, the material of device 500 begins to break down, so that it can be flushed through the toilet and be treated along with other sewerage. The material is chosen such that its breakdown occurs (to the degree that would allow it to pass through the toilet plumbing) within a matter of seconds and significantly less than a minute.

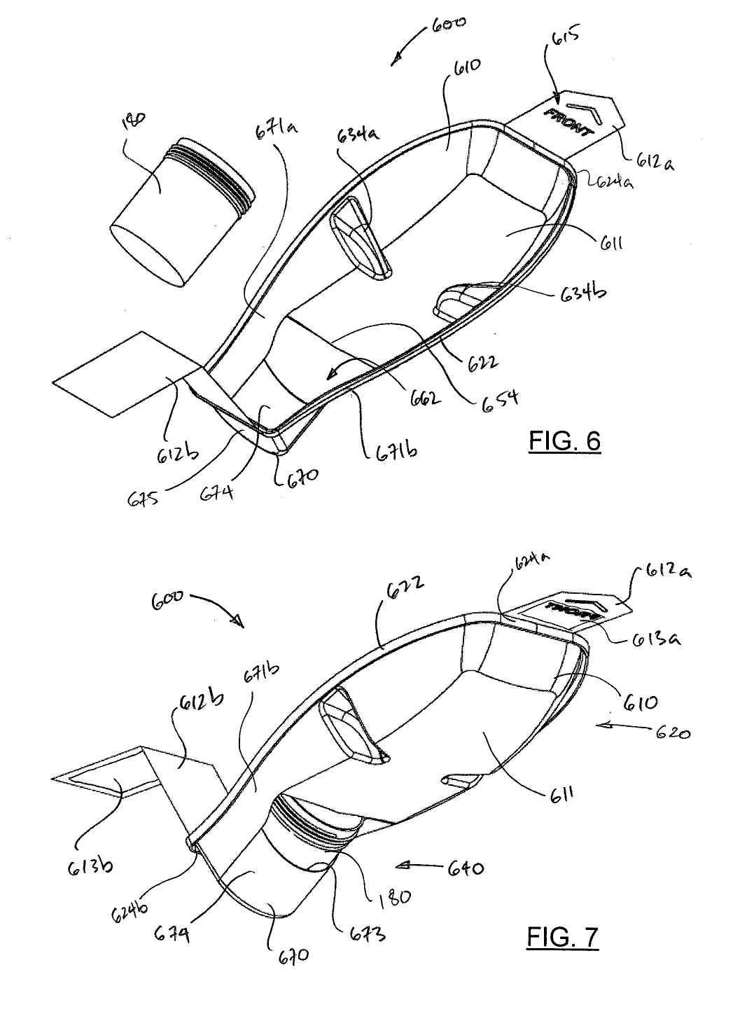

Some embodiments are described by way of example with reference to FIGS. 6 and 7, which show some embodiments of a urine diversion device 600 arranged to divert urine into a standard urine cup 180 when the device 600 is positioned to be suspended at least partly within a toilet bowl (not shown) or other form of toilet. Device 600 is substantially the same as device 500, except that it does not include fluid retention means.

Urine diversion device 600 comprises a body 610 that is shaped to define a basin and has a urine receiving side 620 and an opposed urine collection side 640. Body 610 is generally bowl-shaped on three sides, with upwardly curving side walls extending from a slightly downwardly sloped basin floor 611. The bowl or basin shape of the side walls extends around the urine receiving side 620, but without a corresponding upwardly curved retaining wall at the collection side 640. Instead, collection side 640 is generally arranged to encourage fluid flow from the receiving side 620 into collection cup 180. A cradle 670 extends from the collection side 640 of body 610 to support collection cup 180 to receive fluid flowing under gravity from collection side 640.

Opposed front and back support wings 612a, 612b may extend from opposed front and back rims 624a, 624b (or parts adjacent thereto) of body 610. Support wings 612a, 612b are intended to overlie a toilet seat and optionally adhere thereto by an adhesive 613a, 613b on an underside of the support wings 612a, 612b. Such support wings 612a, 612b may thus provide support to retain body 610 in a suspended position within the toilet bowl while a person is seated on the toilet seat.

In some embodiments, an additional support flap may be coupled to body portion 610, with adhesive on a part thereof to secure the flap to the toilet seat and assist in positioning and supporting body portion 610 relative to the toilet seat. The additional support flap may be formed along a rim 622 of body 610 to one or both lateral sides of a longitudinal centre line of body 610, for example to account for toilet seats that have a central forward gap that may correspond to a centrally positioned flap or support wing 612a. The additional flap may thus be used in tandem with support wing 612a to support body 610 at the front of the toilet.

Both front and back support wings 612a, 612b (and any additional support flap) may be formed of light, flushable materials, such as cellulosic materials, corn starch or other materials that lose structural rigidity or integrity in contact with water-based fluids (i.e. including urine). Light pressure-sensitive adhesive substances that can be easily manually applied and removed may be used to adhere the support wings 612a, 612b and/or a support flap to the toilet seat or, a part of the outside of the toilet bowl.

Support wings 612a, 612b (and any additional support flap) may be integrally formed with, or otherwise coupled, to the body 610 by a suitable adhesive, such as a biodegradable adhesive, for example.

Urine receiving side 620 is generally basin-shaped to receive urine directed toward body 610 when a person is seated on the toilet and device 600 is positioned so that body 610 is generally (hut possibly not entirely) positioned within a volume defined by the toilet seat and the toilet bowl. In this position. The cradle 670 will generally be positioned toward a rear part of the toilet (i.e. toward where a cistern is sometimes located) and a front portion of rim 622 will generally be positioned toward a front of the toilet (as seen by a person sitting normally on the toilet). In such an arrangement, urine passed from a person sitting on the toilet will normally be received within the basin defined by the outwardly curved walls of urine receiving side 620 and sloped basin floor 611, with those walls tending to direct the urine generally inwardly and (at least in part due to a slope of the basin floor 611 of body 610) toward collection side 640.

Flow direction means, for example in the form of upstanding ribs 634a, 634b, may be at least partly provided on body 610 on the urine receiving side 620 and extending toward urine collection side 640 in order to direct urine inwardly and centrally toward collection side 640.

Receiving side 620, including the side walls, basin floor 610 and ribs 634a, 634b, effectively provides diversion means to divert urine received in the body 610 toward the collection side 640.

Collection side 640 of body 610 comprises a fluid ramp or lip 654 positioned on collection side 640 to direct fluid into collection cup 180, as described above.

Cradle 670 is supported by support arms 671a, 671b extending rearwardly and somewhat downwardly from the collection side 640 and is shaped to receive and support part of the curved side wall 181 and base 182 of collection cup 180. Cradle 670 comprises a curved section 674 that is curved to nestingly accommodate the cylindrical side wall 181 of collection cup 180 and a flat section 675 that is shaped and positioned to support the base 182. Cradle 670 is shaped and positioned to receive and support collection cup 180 generally under a centre of gravity of collection cup 180 while collection cup 180 is empty and as it fills with fluid.

An overflow aperture 662 is defined by cradle 670, support arms 671a, 671b and lip 654 so that, once collection cup 180 fills to the point where urine 188 would overflow the cup's upper rim, that urine can flow over the rim and into the toilet bowl through aperture 662. In some embodiments, support arms 671a, 671b may be thickened relative to other parts of body 670 for added support for larger collection cups 180. Cradle 670 is arranged to support collection cup 180 at an angle to the vertical, for example in the order of 70 to 20 degrees from the vertical and optionally between about 30 degrees and about 60 degrees to the vertical.

Once the person has finished passing urine, the collection cup 180 can simply be lifted from cradle 670 and the cap screwed on for provision to a health care professional, for example.

Once urine has been passed and the collection cup 180 has been removed from device 600, the support wings 612a. 612b (and any additional support flap, if appropriate) can be lifted up from the toilet seat and the entire device 600 conveniently disposed of.

In some embodiments, device 600 may be formed of suitable light materials, including paper and plastics materials, that can be readily hygienically disposed of in normal disposal methods.

In some embodiments, body 610 (including integrally formed receiving side 620, collection side 640 and cradle 670) may be formed predominantly of a material that disintegrates or breaks down in water or water-based fluids, such as may be commonly present in a toilet bowl. Such body materials may include, for example, moulded corn starch-based plastic materials, such as Plantic HP1, made by Plantic Technologies of Victoria, Australia.

In such embodiments, an upper surface of body 610 that may come into contact with the urine as it is passed may be coated with a coating that more slowly disintegrates or breaks down than the main body material. Such a coating may include a shellac coating, for example. Body 610 may be uncoated on its underside, for example, where it would not normally come into contact with urine or other water based fluids so that, when device 600 is dropped into a liquid volume at a bottom of the toilet bowl, the material of device 600 begins to break down, so that it can be flushed through the toilet and be treated along with other sewerage.

Device 600 may be used in the collection of a full urine sample (i.e. not discriminating between initial stream and mid-stream urine). Embodiments of device 600 may be configured to have an enlarged and/or reinforced cradle 670 to accommodate a larger collection cup 180.

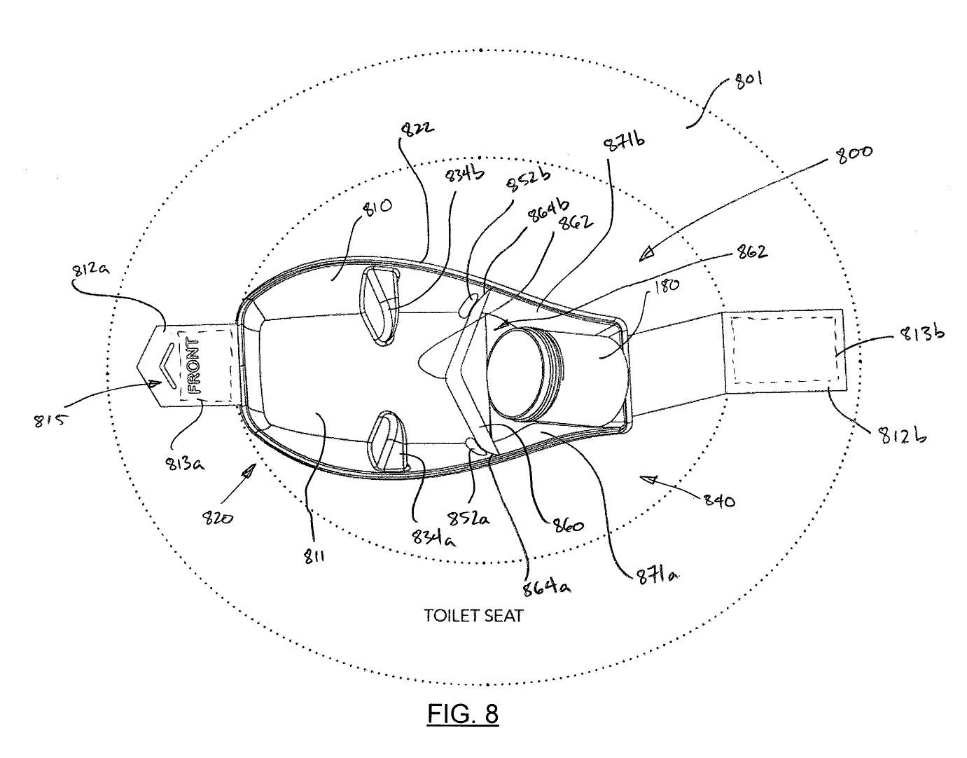

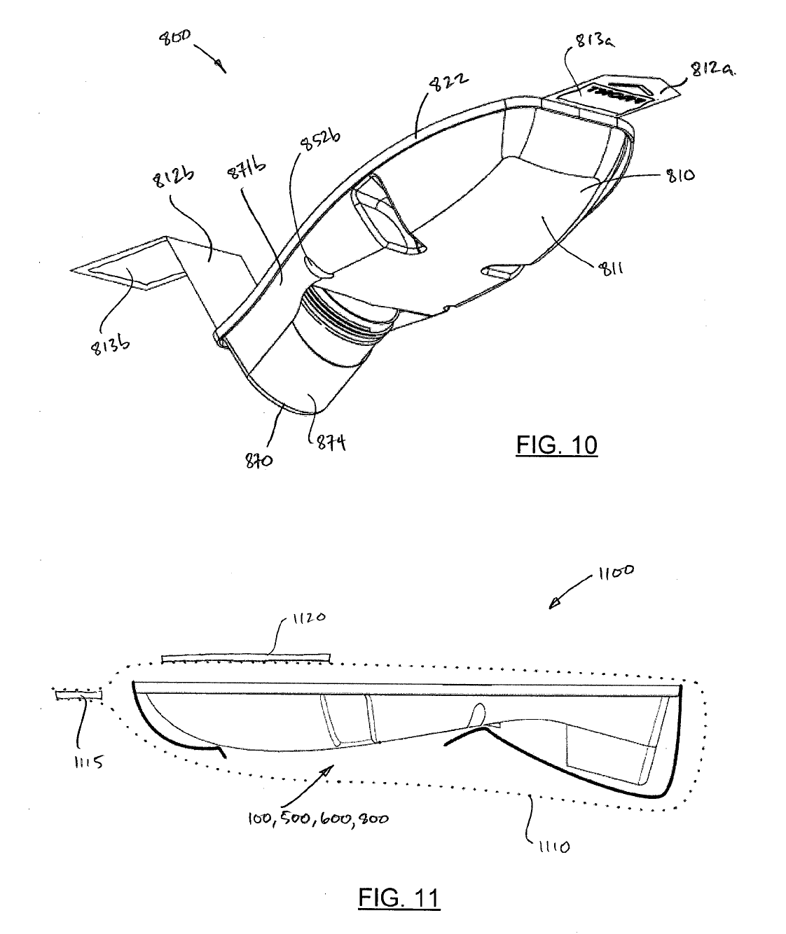

Some embodiments are described by way of example with reference to FIGS. 8, 9A, 9B and 10, showing some embodiments of a urine diversion device 800 arranged to divert urine into a standard urine cup 180 when the device 800 is positioned to be suspended at least partly within a toilet bowl (not shown) or other form of toilet.

Device 800 is substantially the same as device 500, except that it comprises a different means for diverting the initial urine stream and allowing the mid-stream urine to be captured.

Urine diversion device 800 comprises a body 810 that is shaped to define a basin and has a urine receiving side 820 and an opposed urine collection side 840. Body 810 is generally bowl-shaped on three sides, with upwardly curving side walls extending from a slightly downwardly sloped basin floor 811. The bowl or basin shape of the side walls extends around the urine receiving side 820, but without a corresponding upwardly curved retaining wall at the collection side 840. Instead, collection side 840 is generally arranged to encourage fluid flow from the receiving side 820 into collection cup 180 once an initial fluid volume is received within fluid reservoir. 850. A cradle 870 extends from the collection side 840 of body 810 to support collection cup 180 to receive fluid flowing under gravity from collection side 840.

Opposed front and back support wings 812a, 812b may extend from opposed parts of rim 822 (or parts adjacent thereto) of body 810. Support wings 812a, 812b are intended to overlie a toilet seat and optionally adhere thereto by an adhesive (not shown) on an underside of the support wings 812a, 812b. Such support wings 812a, 812b may thus provide support to retain body 810 in a suspended position within the toilet bowl while a person is seated on the toilet seat.

In some embodiments, a front support flap may be coupled to body portion 810, with adhesive on a part thereof to secure flap to the toilet seat and assist in positioning and supporting body portion 810 relative to the toilet seat. The flap may be formed centrally along a forward part of rim 822 of body 810 to one or both lateral sides of a longitudinal centre line 822, for example to account for toilet seats that have a central forward gap that may correspond to a centrally positioned flap. The additional flap may thus be used in tandem with support wing 812a to support body 810 at the front of the toilet.

Both support wings 812a, 812b and any additional support flap may be formed of light, flushable materials, such as cellulosic materials, corn starch or other materials that lose structural rigidity or integrity in contact with water-based fluids (i.e. including urine). Light pressure-sensitive adhesive substances that can be easily manually applied and removed may be used to adhere the support wings 812a, 812b and/or a support flap to the toilet seat or a part of the outside of the toilet bowl. Support wings 812a, 812b and support flap may be integrally formed with or otherwise coupled to the body 810 by a suitable adhesive, such as a biodegradable adhesive, for example.

Urine receiving side 820 is generally basin-shaped to receive urine directed toward body 810 when a person is seated on the toilet and device 800 is positioned so that body 810 is generally (but possibly not entirely) positioned within a volume defined by the toilet seat and the toilet bowl. In this position, the cradle 870 will generally be positioned toward a rear part of the toilet (i.e. toward where a cistern is sometimes located) and front rim 822 will generally be positioned toward a front of the toilet (as seen by a person sitting normally on the toilet). In such an arrangement, urine passed from a person sitting on the toilet will normally be received within the basin defined by the outwardly curved walls of urine receiving side 820 and sloped basin floor 811, with those walls tending to direct the urine generally inwardly and (at least in part due to a slope of the basin floor 811 of body 810) toward collection side 840.

Flow direction means, for example in the form of upstanding ribs 834a, 834b, may be at least partly provided on body. 810 on the urine receiving side 820 and extending toward urine collection side 840 in order to direct urine inwardly and centrally toward collection side 840.

Receiving side 820, including the side walls, basin floor 810 and ribs 834a, 834b, effectively provides diversion means to divert urine received in the body 810 toward the collection side 840.

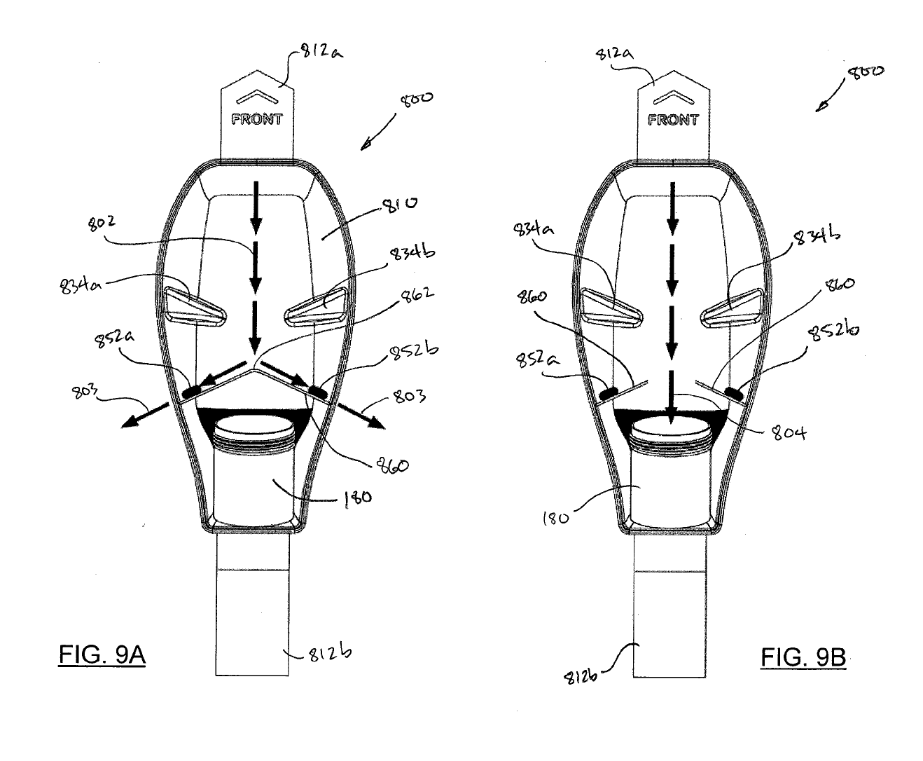

Referring in particular to FIGS. 9A and 9B, use of device 800 in capturing mid-stream urine is further described. As is shown in FIGS. 8, 9A and 9B, device 800 has a barrier element 860 positioned around where the receiving side 820 transitions to the collection side 840 and arranged to provide a barrier to urine flowing into the collection cup 180 from the receiving side 820. Barrier element 860 is of a non-linear configuration, defining an apex 862 positioned generally centrally (i.e. generally in line with a longitudinal axis of the device 800), so as to divert urine impinging on the apex 862 to either lateral side of the apex 862, as illustrated in FIG. 9A. In the described embodiments, barrier element 860 may be approximately chevron shaped. However, other shapes accomplishing a similar diversion function may be employed instead.

The barrier 860 of device 800 is at least partly dissolvable, disintegratable, or otherwise subject to structural breakdown after coming into contact with urine for a time, such as a number of seconds. As shown in FIG. 9A, the barrier element 860 initially diverts the initial urine stream towards side drainage apertures 852a, 852b located at lateral edges of basin 811 where it meets and optionally seals with lateral side edges 864a, 864b of barrier element 860. Thus, for a few seconds, the initial stream of urine is diverted to the drainage apertures 852a, 852b and into the toilet, while the urine that continues to impinge upon barrier element 860 continues to have a dissolving effect. Once the barrier element 860 is sufficiently dissolved or broken down to allow passage of urine therethrough, for example through a central region around where apex 862 was previously positioned, the mid-stream urine is then free to flow through the gap thus created in barrier 860 and to enter into the collection cup 180.

Barrier element 860 may be formed integrally with body 8100r may be provided as an insert to be positioned as described and shown in FIGS. 8, 9A and 9B, extending across the body 810 to provide a fluid flow barrier.

Barrier element 860 is formed of a dissolvable material, for example such as a corn-starch plastic, that begins to break down structurally as soon as it is contacted by water or water-based fluids, such as urine. The time taken to dissolve the central part of the barrier element 860 and subsequently allow the mid-stream urine through is dictated by the thickness of the corn-starch plastic material used (examples of which are described herein) and/or the speed with which the selected material breaks down when contacted with water-based fluid. In order to capture sufficient mid-stream urine, the barrier element 860 should dissolve sufficiently quickly to allow the mid-stream urine into collection cup 180 in less than 10 seconds, preferably less than 5 seconds and more preferably between about 2 and 3 seconds. For this purpose, a thickness of about 0.02 mm to 0.15 mm (for Plantic HP1) may be used, with a thickness between about 0.05 mm and about 0.1 mm being preferred. For other materials, slightly different thicknesses may achieve a similar result.

It should be noted that further embodiments of device 800 may be employed in which barrier element 860 is used in combination with further fluid diversion and/or capture elements, such as those described herein. Additionally, barrier element 860 may comprise or be formed in several pieces. For example, barrier element 860 may include a central barrier portion more forwardly located than separate backwardly and laterally spaced diverting walls, where the central portion is arranged to dissolve within a few seconds, while the remaining lateral diversion walls, which may be formed of a thickened material, remain and define a gap therebetween (originally blocked by the central portion) which allows mid-stream urine to flow into collection cup 180. Further, some embodiments of device 800 may employ barrier element 860 as a trigger element which, when dissolved, causes other fluid diversion structure to allow or promote passage of the urine into collection cup 180.

It should also be noted that embodiments of device 800 may employ a body 810 formed almost entirely of dissolvable material. For such embodiments, the thickness of the material of body 810 may be selected to breakdown substantially later than would occur with the relatively thinner barrier element 860. For example, the thickness of body 810 may be around 0.15 mm to 0.3, 0.4, 0.5 mm or even up to around 1.5 mm.

Collection side 840 of body 810 comprises a fluid ramp or lip 854 positioned on collection side 840 to direct fluid into collection cup 180, as described above.

Cradle 870 is supported by support arms 871a, 871b extending rearwardly and somewhat downwardly from the collection side 840 and is shaped to receive and support part of the curved side wall 181 and base 182 of collection cup 180. Cradle 870 comprises a curved section 874 that is curved to nestingly accommodate the cylindrical side wall 181 of collection cup 180 and a flat section 875 that is shaped and positioned to support the base 182. Cradle 870 is shaped and positioned to receive and support collection cup 180 generally under a centre of gravity of collection cup 180 while collection cup 180 is empty and as it fills with fluid.

An overflow aperture 862 is defined by cradle 870, support arms 871a, 871b and lip 854 so that, once collection cup 180 fills to the point where urine 188 would overflow the cup's upper rim, that urine can flow over the rim and into the toilet bowl through aperture 862. In some embodiments, support arms 871a, 871b may be thickened relative to other parts of body 870 for added support for larger collection cups 180. Cradle 870 is arranged to support collection cup 180 at an angle to the vertical, for example in the order of 70 to 20 degrees from the vertical and optionally between about 30 degrees and about 60 degrees to the vertical.

Once the person has finished passing urine, the collection cup 180 can simply be lifted from cradle 870 and the cap screwed on for provision to a health care professional, for example.

Once urine has been passed and the collection cup 180 has been removed from device 800, the support wings 812a, 812b (and any additional support flap, if appropriate) can be lifted up from the toilet seat and the entire device 800 conveniently disposed of.

In some embodiments, device 800 may be formed of suitable light materials, including paper and plastics materials, that can be readily hygienically disposed of in normal disposal methods.

In some embodiments, body 810 (including integrally formed receiving side 820, collection side 840 and cradle 870) may be formed predominantly of a material that disintegrates or breaks down in water or water-based fluids, such as may be commonly present in a toilet bowl. Such body materials may include, for example, moulded corn starch-based plastic materials, such as Plantic HP1, made by Plantic Technologies of Victoria, Australia.

In such embodiments, an upper surface of body 810 that may come into contact with the urine as it is passed may be coated with a coating that more slowly disintegrates or breaks down than the main body material. Such a coating may include a shellac coating, for example. Body 810 may be uncoated on its underside, for example, where it would not normally come into contact with urine or other water based fluids so that, when device 800 is dropped into a liquid volume at a bottom of the toilet bowl, the material of device 800 begins to break down, so that it can be flushed through the toilet and be treated along with other sewerage.

Referring also to FIG. 11, a package 1100 is shown comprising any one of the urine diversion devices 100, 500, 600, 800 enclosed within packaging material 1110. Packaging material 1110 may be formed of fluid impermeable materials, such as suitable plastics, and may comprise one or more seals 1115 to seal the packaging material 1110 against entry of fluids, such as water or water vapour which, for embodiments of the device that can breakdown in the presence of water based fluids, is necessary to protect the structural integrity of the device prior to use.

Package 1100 may further comprise printed instructions 1120 to illustrate pictorially the intended use of the device 100, 500, 600 or 800. Instructions 1120 may be printed on the packaging material 1110 or a separate substrate fixed to the packaging material 1110. Alternatively or in addition, instructions 1120 may be provided on one or more parts of the device 100, 500, 600, 800.

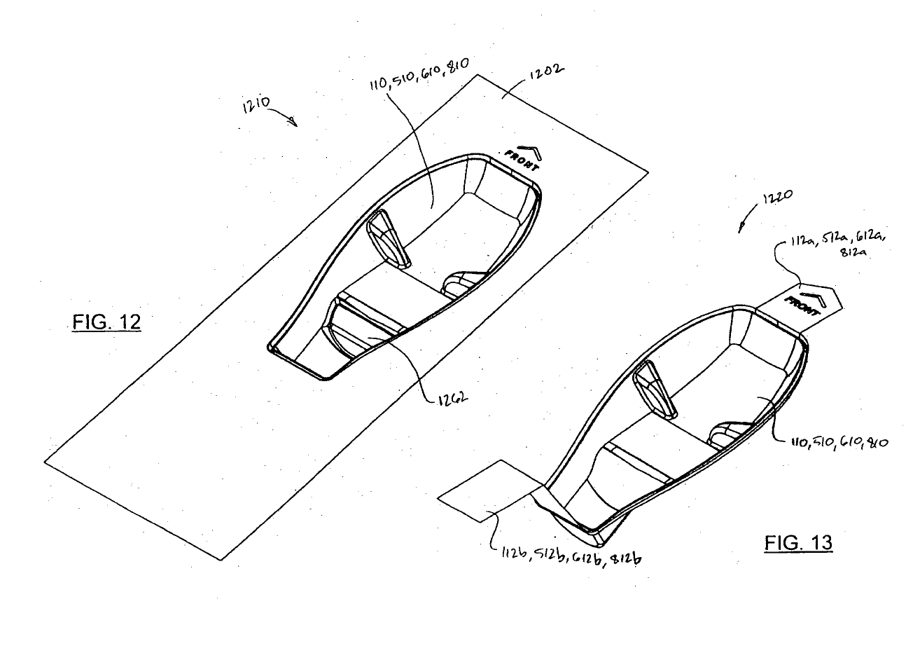

Referring now to FIGS. 12 and 13, a method of manufacturing a urine diversion device (as described herein) is described in further detail, illustrated with reference to device 600 by way of non-limiting example only. FIG. 12 shows a sheet 1202 that has been through a molding step 1210 to form parts of the body 110, 510, 610, 810 of any of the urine diversion devices described herein. Sheet 1202 may be an extruded plastic sheet, for example, having been extruded in an extrusion step performed prior to the molding.

Sheet 1202 may have a generally uniform thickness in the range of about 0.1 mm to about 1.5 mm, for example, although some slight variation in thickness may be present. For embodiments of urine diversion device 100, 500, 600 and 800 that are formed of a dissolvable or disintegratable material, the thickness of sheet 1202 is preferably around 0.1 to 0.4 mm, and optionally 0.1.5 mm to about 0.3 mm to allow the device 100, 500, 600, 800 to breakdown in a toilet quickly enough so that the user does not need to wait appreciably before operating a flushing function of the toilet to dispose of the device 100, 500, 600, 800. Some variation in thickness of sheet 1.202 may occur during the molding and the molding process can be configured to account for this. Thus, the breakdown may occur within a matter of several seconds, such as within about ten 30 seconds, or optionally between about 2 seconds and about 30 seconds and preferably significantly less than 1 minute.

It should be noted, however, that embodiments of the device 100, 500, 600, 800 may be employed without being formed of a dissolvable material or only partly of a dissolvable material. For example, device 800 may comprise a body 810 that is not dissolvable or takes a comparatively long time to dissolve even though barrier element 860 is dissolvable. Additionally, shellac or another surface coating may be used on one or more surfaces of device 100, 500, 600, 800 in order to impede rapid breakdown (when contacted by water or urine) of the part of the body that is coated. This surface coating may be applied after extrusion of the sheet 1202 and optionally after the molding step 1210, but preferably before the cutting step 1220.

In molding step 1210, sheet 1202 is deformed to generally provide most of the contours of the device 100, 500, 600, 800, but the support wings 112a/b, 512a/b, 612a/b and 812a/b are not yet separately formed as they will be cut from sheet 1202 in cutting step 1220.

Additionally, molded sheet 1202 comprises one or more sheet portions 1262 to be removed in order to allow the body of 1110, 510, 610, 810 to define apertures 162, 562, 662 and 862 for overflow drainage, as well as further apertures 152, 552 and 852, where the initial urine stream is to be captured and/or diverted so that the midstream can flow into the urine collection cup 180. In the cutting step illustrated by 1220 in FIG. 13, portions 1262 are cut from the body 110, 510, 610, 810.

Either before or after the cutting step 1220, but after the extrusion of sheet 1202 and preferably after the molding, adhesive substances are preferably applied to the portions of sheet 1202 that are to form (or have been cut to form) the support wings 112, 512, 612, 812. This is in order to provide adhesive pads 513, 613 and 813 to allow the device 100, 500, 600, 800 to be coupled to a seat or rim or other support surface to be partially within a toilet bowl or other interior toilet volume for use in the manner previously described and illustrated by way of example in FIG. 8. The application of the adhesive may be performed at or around the same time as the application of the surface coating, if any such coating is applied.

Where a flap 160 or 560 is to be used in the device 100, 500 being made according to the described process, this flap 160 or 560 may be molded in a separate part of sheet 1202, and the flap 160, 560 is then cut separately (but optionally simultaneously) to the cutting of the device body 110 or 510. The cutting may include cutting the flap 160, 560 to define a tongue or extension portion that extends from a line along where the flap 160, 560 is to hinge or pivot relative to the body 110, 510. While performing the cutting, the body 110, 510 may be further cut to define a narrow slit to receive the tongue of the flap 160, 560. This separate flap 160, 560 can then be assembled with the body 110, 510 so that the tongue extends through the slit and the flap 160, 560 is free to hinge or pivot as the reservoir 150, 160 fills from fluid passing across the flap 160, 560. In other embodiments, the flap 160, 560 may be integrally formed with the body or may be adhered or otherwise coupled thereto.

If the described process is used to make the device 100, 500, 600, 800, then the body and cradle are generally formed of a single layer of (sheet) material, at least prior to application of any adhesive or shellac.

While the embodiments shown and described herein illustrate intended use and operation of the urine diversion device 100, it should be understood that some variations on the specific arrangements falls within the scope of the described embodiments. In particular, the size, proportion, composition and configuration of described elements and features may vary among embodiments, without departing from the spirit and scope of the invention. Embodiments include various combinations of features described herein and accordingly the drawings and the appended claims should not be construed as an exhaustive list of inventive features for which patent protection may be sought in this application or any application derived from it.

For example, flap 160 or 560 may be configured in a non-hinged or differently hinged manner that still allows movement between the lowered and raised positions. Additionally, support arms 171a/171b, 571a/571b, 671a/671b and 871a/871b may alternatively be formed as a single cradle support arm. Further, flap 160 or 560 and fluid reservoir 150 or 550 may be differently shaped while accomplishing the described functions.

Further, the support wings 112a/b, 512a/b, 612a/b and 812a/b may be formed with various shapes and/or dimensions and may be supplemented by additional wings and/flaps for added support and/or stability, provided that they serve the minimum function of stably supporting the device in a position within (or partly within) the toilet so as to receive urine directed forwardly and somewhat downwardly from a person sitting (facing forwardly) on the toilet, while not being so low as to contact or interfere with the toilet bowl or fluid resting therein. Such support wings or flaps may be positioned at various positions around the rim and/or seat.

As shown in FIGS. 5A to 13, at least one of the support wings 512a/b, 612a/b and 812a/b may be formed with an indication 515, 615, 815 of orientation for intended/recommended use, such as a printed or embossed arrow and/or word, such as “front” or “back”. This indication may also be provided on either or both of support wings 112a and 112b and/or on flap 114. The indication of orientation may be formed during the molding step if it is an embossed indication.

In the described embodiments, rim 122, 522, 622 and 822 may be formed with a structural variation, such as a fold, crease or angle, extending around the upper outer extent of body 110, 510, 610, 810 to lend greater structural rigidity to the device 100, 500, 600, 800.

It should be noted that, while the device is particularly discussed as being positionable at least partly within a toilet, this is intended to also include positioning of the device within related devices that might not be standard toileting devices. Thus, the term toilet as used herein is intended to include conventional toilets defining a bowl within which waste is to be received, as well as bides, toilets that are squatted over rather than sat upon, camp toilets, commodes, outdoor toilets, raised toilet seats and frames, bedpans and related arrangements that have some kind of support structure upon which the described support wings 112a/b, 512a/b, 612a/b and 812a/b (and any other support or stabilisation flaps) can be rested and/or temporarily affixed while the device is suspended at a somewhat lowered position in an opening or aperture defined by the support structure. The term toilet is thus not intended to be construed in a limiting fashion.

While embodiments are described herein in specific detail, it is to be understood that such embodiments are described by way of example and are not to be construed to be limiting with respect to equivalents or to limit the scope of the invention.

Throughout this specification and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1-38. (canceled)

39. A urine diversion device to be positioned at least partly in a toilet, the device comprising:
a body defining a basin and having a collection side, the basin shaped to direct urine received in the basin toward the collection side;
support elements on the body to support the body in a suspended manner at least partly in the toilet; and
a cradle defined by the body at the collection side to support a urine receptacle to receive urine directed toward the collection side;
wherein the device is disposable in the toilet.

40. The device of claim 39, wherein the device comprises material that is dissolvable or disintegratable in water and water-based liquids.

41. The device of claim 40, wherein the body and the cradle are formed of the material.

42. The device of claim 40, wherein the material is a corn-starch-based material.

43. The device of claim 39, further comprising means to divert an initial stream of urine from entering the urine receptacle.

44. The device of claim 43, wherein the means to divert is arranged to allow urine after the initial stream to enter the urine receptacle.

45. The device of claim 43, wherein the means to divert comprises a dissolvable harrier.

46. The device of claim 45, wherein the dissolvable barrier is configured to dissolve within about 5 seconds from when it is contacted by the initial stream of urine.

47. The device of claim 46, wherein the dissolvable barrier is configured to dissolve within about 3 seconds from when it is contacted by the initial urine stream.

48. The device of claim 45, wherein the dissolvable barrier has a non-linear shape defining an apex.

49. The device of claim 48, wherein the apex is centrally positioned to direct the initial stream towards at least one drainage aperture.

50. The device of claim 49, wherein the at least one drainage aperture comprises two apertures, one aperture positioned toward each lateral side of the body.

51. The device claim 39, wherein the body defines at least one drainage aperture to drain excess urine.

52. The device of claim 51, wherein the at least one drainage aperture comprises a central aperture defined by the body to allow drainage of overflow from the urine receptacle.

53. The device of claim 45, wherein the dissolvable barrier is integrally formed with the body.

54. The device of claim 45, wherein the dissolvable barrier is formed of a corn starch-based material.

55. The device of claim 45, wherein the dissolvable barrier has a thickness between about 0.02 mm and about 0.15 mm.

56. The device of claim 55, wherein the dissolvable barrier has a thickness between about 0.05 mm and about 0.1 mm.

57. The device of claim 39, wherein the support elements each have an adhesive substance on a face thereof to adhere the respective support element to a rim or seat of the toilet.

58. The device of claim 39, wherein the support elements comprise opposed first and second support elements, wherein the second support element is longer than the first support element.

59. The device of claim 58, wherein at least one of the first and second support elements comprises an indicator of intended positioning of the device in relation to the toilet.

60. A urine diversion device to be positioned at least partly in a toilet, the device comprising:
a body defining a basin and having a collection side, the basin shaped to allow urine received in the basin to flow toward the collection side;
support elements on the body to support the body in a suspended manner at least partly in the toilet;
a receptacle support at the collection side to support a urine receptacle to receive urine flowing to the collection side; and
a dissolvable barrier element to act as a barrier to urine flowing toward the collection side.

61. The device of claim 60, wherein the dissolvable barrier is configured to dissolve within about 5 seconds from when it is contacted by the initial stream of urine.

62. The device of claim 61, wherein the dissolvable barrier is configured to dissolve within about 3 seconds from when it is contacted by the initial urine stream.

63. The device of claim 60, wherein the dissolvable barrier has a non-linear shape defining an apex.

64. The device of claim 63, wherein the apex is centrally positioned to direct the initial stream towards at least one drainage aperture.

65. The device of claim 64, wherein the at least one drainage aperture comprises two apertures, one aperture positioned toward each lateral side of the body.

66. The device of claim 54, wherein the body defines at least one drainage aperture to drain excess urine.

67. The device of claim 66, wherein the at least one drainage aperture comprises a central aperture defined by the body to allow drainage of overflow from the urine receptacle.

68. The device of claim 60, wherein the dissolvable barrier is integrally formed with the body.

69. The device of claim 60, wherein the dissolvable barrier is formed of a corn starch-based material.

70. The device of claim 60, wherein the dissolvable barrier has a thickness between about 0.02 mm and about 0.15 mm.

71. The device of claim 70, wherein the dissolvable barrier has a thickness between about 0.05 mm and about 0.1 mm.

72. A urine diversion device to be positioned at least partly in a toilet, the device comprising:
a body defining a basin and having a collection side, the basin shaped to allow urine received in the basin to flow toward the collection side;
support elements on the body to support the body in a suspended manner at least partly in the toilet;
a receptacle support at the collection side to support a urine receptacle to receive urine flowing to the collection side; and
wherein at least the body is formed of a molded sheet having a thickness between 0.1 mm and about 1.5 mm.

73-90. (canceled)
